(19) United States
(12) Patent Application Publication
(10) Pub. No.: US 2018/0214453 A1
(43) Pub. Date: Aug. 2, 2018

(54) RIVAROXABAN PHARMACEUTICAL COMPOSITIONS

(71) Applicant: INTERQUIM, S.A., Sant Cugat Del Vallès (ES)

(72) Inventor: Berta FERNÁNDEZ MOLLAR, Sant Cugat Del Vallès (ES)

(73) Assignee: INTERQUIM, S.A., Sant Cugat Del Vallès (ES)

(21) Appl. No.: 15/742,166

(22) PCT Filed: Aug. 4, 2016
(86) PCT No.: PCT/EP2016/068619

§ 371 Date: Jan. 5, 2018

(30) Foreign Application Priority Data
Aug. 5, 2015 (EP) 15179820.4

(51) Int. Cl. A61K 31/5377; A61K 9/20

(52) U.S. Cl. CPC A61K 31/5377; A61K 9/2095; A61K 9/2031

(57) ABSTRACT

A rivaroxaban immediate release tablet comprising a non-ionic surfactant obtainable by using a dry process (i.e. no solvent is used during the manufacturing of the core tablet) which rapidly releases the drug as well as shows high stability and its fast and fed bioavailability is only attributable to the rivaroxaban behaviour.

### FILED OF THE INVENTION

The present invention is in the field of pharmaceutical compositions, specifically in the field of tablet compositions and more specifically in the field of immediate release tablet compositions.

### BACKGROUND OF THE INVENTION

Rivaroxaban was first disclosed in WO0147919 A1, has the systematic name 5-chloro-N-({(5S)-2-oxo-3-[4-(3-oxomorpholin-4-yl)phenyl]-1,3-oxazolidin-5-yl}methyl)thiophene-2-carboxamide and the following chemical structure:

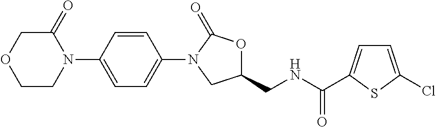 [SMILES: O=C(NC[C@H]1CN(c2ccc(N3CCOCC3=O)cc2)C(=O)O1)c1ccc(Cl)s1]

Three different polymorphic forms of rivaroxaban have been described in WO07039132 A1.

Rivaroxaban is an orally active factor Xa inhibitor marketed for the treatment or prevention of atherothrombotic events, prevention of venous thromboembolism, prevention of stroke and systemic embolism, treatment and prevention of deep vein thrombosis or treatment and prevention of pulmonary embolism. Rivaroxaban is marketed as Xarelto® in 2.5, 10, 15 and 20 mg strengths immediate release tablets.

Rivaroxaban is practically insoluble in water (according to WO05060940 A2 it has a solubility of about 7 mg/L) and has high permeability, thus being a BCS class II compound. According to the CHMP assessment report for Xarelto®, rivaroxaban is slightly soluble in organic solvents (1.0-10.0 g/L).

Due to the low solubility of rivaroxaban, the use of small particle size is required in order to obtain bioavailable compositions. Small particle size may adversely impact blend flowability, which makes the manufacturing process of the rivaroxaban pharmaceutical composition difficult, especially with regard to reproducibility and content uniformity. Several strategies to improve the solubility of rivaroxaban have been proposed.

One strategy to improve the solubility is moist granulation:
WO05060940 A2 discloses rivaroxaban formulations prepared using granules comprising rivaroxaban in hydrophilized form that are prepared by moist granulation.
IN03369CH2013 A discloses rivaroxaban formulation including from 5 to 90% w/w of disintegrant prepared by moist granulation.
CN104337787 A discloses rivaroxaban formulations expressed in w/w percentages comprising rivaroxaban 3-30%, disintegrant 7-50%, diluent 45-90%, wetting agent 0.1-5% and lubricant 0.1-1% prepared using wet granulation process.
CN103550165 A discloses rivaroxaban formulations prepared by a wet granulation process.

The preparation of rivaroxaban formulation using wet granulation processes is not desirable due to the length of the process and its reduced yield due to drying and sieving processes.

Other strategies include the use of metastable or amorphous crystalline forms that are known to have higher solubility than the thermodynamically stable crystalline form:
WO07039122 A2 discloses rivaroxaban formulations comprising rivaroxaban in amorphous form or thermodynamically metastable crystal modification.
WO09049820 A2 discloses rivaroxaban formulations comprising at least one solid substance at room temperature, the thermodynamically preferred crystalline form of rivaroxaban, cellulose and/or cellulose derivatives, as well as optionally a surface-active substance, wherein more than 50% of rivaroxaban in the formulation is in amorphous mixed phase as determined by X-ray diffractometry.
CN103705520 A discloses rivaroxaban formulations prepared by spray drying, which is a known technique to prepare amorphous forms.

The use of metastable or amorphous crystalline forms is not desirable due to possible change into the stable crystalline form of rivaroxaban, which will result in an undesirable modification in the dissolution profile during storage and thus in a modification of the behaviour of the pharmaceutical composition during time.

Another strategy proposed in the art is the use of a complex step in the preparation of the formulation that helps to improve the solubility of rivaroxaban:
WO10146179 A2 or WO11042156 A1 disclose hot melt rivaroxaban formulations.
WO13022924 A1 discloses rivaroxaban formulations with inert cores coated with a drug layer.
WO14191446 A1 discloses rivaroxaban formulations whose preparation includes the step of milling rivaroxaban together with a hydrophilic binder.
CN103550166 A discloses a rivaroxaban microsphere formulation comprising rivaroxaban and a water-soluble polymer carrier, said carrier comprising a water-soluble polymer selected form hydroxypropylmethyl cellulose, polyethylene glycol, polyvinyl polyvinylpyrrolidone, cellulose polymers and combinations thereof.

The use of hot melt process, coated granules, milling with excipients or microspheres formulations imply complex, lengthy and costly processes; therefore they are not desirable.

Another strategy proposed in the art is the use of substances in the preparation of the formulation that help to improve the solubility of rivaroxaban:
WO10003641 A1 discloses pharmaceutical compositions comprising (a) rivaroxaban, (b) a solubilizer and (c) a pseudo-emulsifier as excipients. The use of pseudo emulsifiers is not desirable due to its sticky properties and tendency to adhere to the punch and die, as well as to the rest of equipment employed in the manufacturing process, which is very problematic when the process is scaled up.
CN104055743 A discloses rivaroxaban formulations comprising carbomer which improves the dissolution rate and citric acid which improves the stability of the formulation. The use of carbomer is not desirable because this excipient when dissolved in water and turned into neutral o basic conditions forms gels that slow the rivaroxaban release. Considering this, the behaviour of rivaroxaban tablets containing carbomer will have a longer release time when administered with food than under fast conditions (food effect).
CN104666262 A discloses rivaroxaban formulation prepared using rivaroxaban dissolved in diethylene glycol monoethyl ether and then converted into a tablet. The use of a liquid in the preparation of a tablet is not desirable, not only because special equipment is required, but also because it is more difficult to handle.

Therefore there is a need in the art to obtain a rapid release rivaroxaban pharmaceutical composition which is stable that is short, cheap and easy to manufacture and is not affected by a food effect.

### SUMMARY OF THE INVENTION

The present invention relates to an immediate release tablet comprising
rivaroxaban,
a non-ionic surfactant and
optionally one or more pharmaceutically acceptable excipients

obtainable by using a dry process.

Another object of the invention is the tablets of the present invention for the treatment or prevention of atherothrombotic events, prevention of venous thromboembolism, prevention of stroke and systemic embolism, treatment and prevention of deep vein thrombosis or treatment and prevention of pulmonary embolism.

Another object of the invention is a process for the preparation of the tablets of the present invention, which comprises the following steps:

a) mixing rivaroxaban with at least the non-ionic surfactant,

b) compacting the mixture of step a),

c) sieving the compacted mixture of step b),

d) optionally adding the rest of the excipients and mixing, and

e) compressing the resulting mixture of steps c) or d) into a tablet,

wherein no liquid solvent is used in the process ending in step e).

Another object of the invention is a process for the preparation of the tablets of the present invention, which comprises the following steps:

a) mixing rivaroxaban with at least the non-ionic surfactant,

b) optionally adding the rest of the excipients and mixing, and

c) compressing the resulting mixture into a tablet,

wherein no liquid solvent is used in the process ending in step c).

The inventors have found that using a dry process (i.e. using no solvent during the manufacture of the core of the tablet) and a non-ionic surfactant, a rivaroxaban tablet is easily prepared which rapidly releases the drug, shows high stability and its fast and fed bioavailability is mainly attributable to the different rivaroxaban solubility in aqueous and lipophilic environments.

### Definitions

In the context of the present invention, an immediate release tablet is a tablet that releases at least 85% of the rivaroxaban content in 45 min using the preferred dissolution method. The preferred dissolution method, according to the Dissolution Methods Database of the US FDA, is as follows:

| Tablet | USP | Speed | | Volume |
|---|---|---|---|---|
| strength | apparatus | (RPMs) | Medium | (mL) |
| 2.5 and 10 mg | II (Paddle) | 75 | Acetate Buffer | 900 |
| | | | pH 4.5, 0.2% SDS* | |
| 15 and 20 mg | II (Paddle) | 75 | Acetate Buffer | 900 |
| | | | pH 4.5, 0.4% SDS* | |

*sodium dodecyl sulfate

Compressing is applying enough force to a mixture to convert that mixture into a tablet. This is sometimes referred as tabletting. Typically, the force applied in compression ranges from 3 kN to 100 kN, more commonly from 4 kN to 75 kN and most commonly, from 10 kN to 30 kN.

Compacting a mixture is a densification and agglomeration process by the direct application of a force between 0.1 KN and 15 KN. The powder morphology is modified through the use of dry compaction forces. This process can be carried out with a roller compactor, a tablet press or the like.

A pharmaceutically acceptable excipient is a component of a pharmaceutical composition or formulation which has one or more functions and is suitable to be administered to any animal including mammals and humans. Some of the functions that the excipient may perform are: surfactant, diluent, binder, disintegrant, glidant, lubricant, coating, colorant, flavouring agent, sweetener, and the like.

A non-ionic surfactant is an amphiphilic molecule having a hydrophobic part and a hydrophilic part, which allows it to be a surface active molecule, that is substantially non-ionized (i.e. uncharged) in water in neutral pH. Suitable non-ionic surfactants include, but are not limited to, block copolymers of ethylene and propylene oxide, fatty acid esters, medium-chain triglycerides, cyclodextrins, polyethylene glycols, sorbitan alkyl esters, polyoxyethylene sorbitan fatty acid esters, polyoxylglycerides, polyoxyethylene fatty alcohol ethers, polyoxyethylene castor oil derivatives, polyoxyethylene alkyl esters, polyvinyl caprolactam-polyvinyl acetate-polyethylene glycol graft copolymer or polypropylene glycols esters, mixtures thereof and the like.

Block copolymers of ethylene and propylene oxide known as poloxamers are copolymers composed by a central hydrophobic chain of polyoxypropylene flanked by two hydrophilic chains of polyoxyethynene. Poloxamers are defined by a three-digit number, wherein the first two digits multiplied by 100 gives the approximate molecular mass of the polyoxypropylene core, and the last digit multiplied by 10 gives the percentage polyoxyethylene content. Examples of suitable poloxamers are poloxamer 124, 188, 237, 331, 338, 407 and the like. Examples of commercial block copolymers of ethylene and propylene oxide are Kolliphor® P188, Kolliphor® P188 micro, Kolliphor® P237, Kolliphor® P338, Kolliphor® P407, Kolliphor® P407 micro, Synperonic® PE/F 127, Synperonic® PE/F 68, Synperonic® PE/F 108, Synperonic® PE/F 44 or Synperonic® PE/F 101.

Fatty acid esters are esters of one fatty acid (fatty acids are linear or branched, saturated or insaturated carboxylic acids from 10 to 22 carbons) esterified to an alcohol moiety, including polyols such as glycol. Examples of suitable fatty acid ester are glyceryl monooleate (such as Capmul GMO-50 or CremerCOOR® GMO 90), glyceryl monostearate (such as Capmul GMS-50K or Cutina® GMS V), glyceryl caprylate (such as Capmul MCM C8 or Capmul MCM C8 EP), glyceryl caprate (such as Capmul MCM 010) and the like.

A medium-chain triglyceride is a glycerol moiety esterified with three medium-chain fatty acids. Medium chain fatty acids are considered fatty acids with of 6 to 12 carbons atoms in length, such as caproic acid, enanthic acid, caprylic acid, pelargonic acid, capric acid, undecylic acid or lauric acid. Examples of medium-chain triglycerides are caprylic triglyceride, caprylic/capric triglyceride and like. Commercial examples of medium-chain triglyceride are Miglyol® 812, Bergabest® MCT Oil 60/40, Bergabest® MCT Oil 70/30, Bergabest® MCT Oil 8/100, Captex® 355, Captex® 355 EP/NF, Captex® 355 EP/NF/JPE, Captex® 355 low C6, Crodamol® GTCC-PN, Labrafac® CC, Labrafac® Lipophile WL1349; Myritol® 318, Myritol® 812, Myritol® 312, Neobee® 895, Neobee® 1053 or Waglinol® 3/9280.

Cyclodextrins is a family of molecules made up of sugar molecules bound together to form a ring. Examples of cyclodextrines are α-cyclodextrin, β-cyclodextrin, γ-cyclodextrin, 2-hydroxypropyl-β-cyclodextrin, methyl-β-cyclodextrin, dimethyl-β-cyclodextrin, 2-hydroxyethyl-β-cyclodextrin, sulfobutylether-β-cyclodextrin, trimethyl-β-cyclodextrin and the like. Examples of commercial cyclodextrins are Cavitron W7 HP5 Pharma cyclodextrin, Cavitron W7 HP7 Pharma cyclodextrin, Cavamax W6 Pharma, Cavamax W7 Pharma, Cavamax W8 Pharma or Klepsose® DC.

Polyethylene glycols, also known as macrogols, PEG or polyethylene oxides, are polymers of ethylene oxide of different length. The different grades of polyethylene glycols are referred as PEG followed by a number stating the average molecular weight. Examples of suitable polyethylene glycols are PEG 200, PEG 300, PEG 400, PEG 540, PEG 600, PEG 900, PEG 1000, PEG 1450, PEG 1540, PEG 2000, PEG 3000, PEG 3350, PEG 4000, PEG 4600, PEG 8000 and the like. Examples of commercial polyethylene glycols are Kollisolv® PEG 300, Kollisolv® PEG 400, Renex™ PEG 400, Renex™ PEG 1500, Renex™ PEG 4000 and Renex™ PEG 6000.

Sorbitan alkyl esters are molecules derived from sorbitan esterified with one or more fatty acid. Suitable sorbitan alkyl esters are sorbitan monoisostearate, sorbitan monolaurate, sorbitan monooleate, sorbitan monopalmitate, sorbitan monostearate, sorbitan sesquiisostearate, sorbitan sesquioleate, sorbitan trilaurate, sorbitan trioleate, sorbitan tristearate and the like. Commercial examples are Sorbester® P17, Sorbirol® L, Hodag®, Lamesorb®, Liposorb®, Montane®, Nikkol®, Polycon®, Sorgon® or Span® 20, Span® 40, Span® 60, Span® 65, Span® 80, Span® 85 or SP Span® 60 MBAL.

Polyoxyethylene sorbitan fatty acid esters are molecules derived from polyethylenglycolated sorbitan moieties esterified with one or more fatty acid. Suitable polyoxyethylene sorbitan fatty acid esters are polysorbate 20 (polyoxyethylene 20 sorbitan monolaurate), polysorbate 21 (polyoxyethylene (4) sorbitan monolaurate) polysorbate 40 (polyoxyethylene 20 sorbitan monopalmitate), polysorbate 60 (polyoxyethylene 20 sorbitan monostearate), polysorbate 61 (polyoxyethylene (4) sorbitan monostearate), polysorbate 65 (polyoxyethylene 20 sorbitan tristearate), polysorbate 80 (polyoxyethylene 20 sorbitan monooleate), polysorbate 81 (polyoxyethylene (5) sorbitan monooleate), polysorbate 85 (polyoxyethylene 20 sorbitan trioleate) or polysorbate 120 (polyoxyethylene 20 sorbitan monoisostearate). There two ways of referring to this family of compounds, “polysorbates” plus a number or “polyoxyethylene” plus a number and the fatty acid present. The number following ‘polysorbate’ is related to the type of fatty acid associated with the polyoxyethylene sorbitan part of the molecule. Monolaurate is indicated by 20, monopalmitate is indicated by 40, monostearate by 60 and monooleate by 80, when the second digit is a zero polysorbates includes a total of 20 units of oxyethylene —(CH₂CH₂O)—, when the second digit is not a zero polysorbates includes a total of oxyethylene —(CH₂CH₂O)— units different from 20. In the second way of referring to this compounds the number following ‘polyoxyethylene’ refers to the total number of oxyethylene —(CH₂CH₂O)— units found in the molecule. Commercial examples are Tween® 20, Tween® 21, Tween® 40, Tween® 60, Tween® 61, Tween® 65, Tween® 80, Tween® 81, Tween® 85, Kolliphor® PS 20, Kolliphor® PS 60 or Kolliphor® PS 80.

Polyoxylglycerides are mixtures of monoesters, diesters, and triesters of glycerol, and monoesters and diesters of polyethylene glycols (PEG). The polyoxyethylene moiety is commonly referred to as macrogol or polyoxyl and a digit giving the number of ethylene oxide units. Suitable examples of polyoxylglycerides are caprylocaproyl macrogolglycerides (such as Labrasol®), lauroyl macrogolglycerides (such as Gelucire® 44/14, and the like), linoleoyl macrogolglycerides (such as Labrafil® M2125CS, oleoyl macrogolglycerides (such as Labrafil® M1944CS), polyethylene glycol monocetyl ester, stearoyl macrogolglycerides (such as Gelucire® 50/13) and the like.

Polyoxyethylene fatty alcohol ethers are molecules made up of a fatty alcohol etherified to a polyethylene glycol of a given length, such as polyethylene glycol monooleyl ether, polyethylene glycol monolauryl ether, polyethylene glycol monostearyl ether and the like. Commercial examples are members of the Brij® family such as Brij® O2, Brij® O10, Brij® CS17, Brij® S2, Brij® S10, Brij® S2, Brij® C20, Brij® L23, Brij® IC20, Brij® LT3 or Brij® CS25.

Polyoxyethylene castor oil derivatives are molecules of castor oil etherified with polyethylene glycol. Examples of polyoxyethylene castor oil derivatives are macrogolglycerol ricinoleate, polyoxyl 35 castor oil, macrogolglycerol hydroxystearate, PEG-40 hydrogenated castor oil, PEG-35 castor oil, polyoxyl castor oil (such as Kolliphor® RH 40 or Kolliphor® EL/ELP) and the like.

Polyoxyethylene alkyl esters are esters of polyoxyethylene alcohol and an alkyl carboxylic acid. Suitable polyoxyethylene fatty acid esters are polyoxyethylene stearates esters such as polyoxyl 2, 4, 6, 8, 12, 20, 30, 40, 50, 100 or 150 stearates; polyoxyethylene 12, 32 and 150 distearates and the like. Commercial examples are Protamate® 200-OC (PEG-4 oleate), Protamate® 200-DPS (PEG-4 stearate), Protamate® 300-DPS (PEG-6 stearate), Protamate® 400-DPS (PEG-8 stearate), Protamate® 600-DPS (PEG-12 stearate), Protamate® 1540-DPS (PEG-40 stearate), Protamate® 2000-DPS (PEG-40 stearate), Protamate® 4400-DPS (PEG-100 stearate), Protamate® 200-ML (PEG-4 laurate), Protamate® 400-ML (PEG-8 laurate), Protamate® 600-ML (PEG-15 laurate), Protamate® 400-DO (PEG-8 dioleate), Protamate® 200-DS (PEG-4 distearate), Protamate® 400-DS (PEG-8 distearate), Protamate® 600-DS (PEG-12 distearate), Protamate® 6000-DS (PEG-150 distearate), Protamate® 200-DL (PEG-8 dilaurate) or Protamate® 400-DL (PEG-8 dilaurate).

Polyvinyl caprolactam-polyvinyl acetate polyethylene glycol graft copolymer is a copolymer consisting of a main polymer chain or backbone (polyethylene glycol) covalently bound to one or more side chains (copolymer of vinylcaprolactam and vinyl acetate) such as a graft-copolymer of PEG 6000 with vinylcaprolactam and vinyl acetate commercially available as Soluplus®.

Polypropylene glycols esters are molecules made up of polypropylene glycol moiety esterified with fatty acids. Suitable examples are propylene glycol dicaprylate, propylene glycol dicaprate or propylene glycol dicaprylocaprate (such as Labrafac™ PG) and the like.

A diluent is an inert pharmaceutically acceptable excipient that provides bulk to the pharmaceutical composition, facilitates the manufacturing process of the dosage form as well as improves the uniformity of content of the active ingredient in the composition. Suitable examples of diluents are: microcrystalline cellulose, lactose (including but not limited to lactose USP, anhydrous lactose USP and spray-dried lactose USP), starch (including but not limited to maize starch, dry starch and directly compressible starch and hydrolyzed starch (including but not limited to Celutab®) sucrose-based diluents (including but not limited to sucrose, confectioner's sugar and sugar spheres NF), dextrose (including but not limited to Cerelose® and dextrose monohydrate), dicalcium phosphate (including but not limited to dicalcium phosphate trihydrate), monobasic calcium sulfate monohydrate, calcium sulfate dihydrate, calcium lactate trihydrate (including but not limited to calcium lactate trihydrate granular NF), calcium carbonate, dextrates (e.g., Emdex®), hydrolyzed cereal solids (including but not limited to Matron products and Mor-Rex), amylose, Recel, powdered cellulose (including but not limited to Elcema®), glycine, bentonite and the like.

A binder is a pharmaceutically acceptable excipient that holds the components of a solid pharmaceutical composition together, such as granules or tablets. Suitable binders are: starch (including but not limited to maize starch and pregelatinized starch), gum acacia, gum arabica, gelatine, cellulose and derivatives thereof (including but not limited to cellulose esters, cellulose ethers and hydroxypropylcellulose) and the like.

A disintegrant is a pharmaceutically acceptable excipient that included in solid pharmaceutical forms, such as tablets or granules, facilitate its break up or disintegration in an aqueous environment. Suitable disintegrants are starches (including but not limited to sodium starch glycolate, corn starch, potato starch, maize starch, modified starches and pregelatinized corn starches (including but not limited to National 1551 and National 1550)), clays (including but not limited to bentonite, bentonite magma, purified bentonite, kaolin, ball clay, common clay, magnesium aluminium silicate, magnesium trisilicate and shale, and fire clay), celluloses (including but not limited to purified cellulose, methylcellulose and carmellose sodium (also known as sodium carboxymethylcellulose), cross-linked cellulloses, such as cross-linked carmellose (croscarmellose) and its salts, including sodium croscarmellose), alginates, gums (such as agar, guar, locust bean, karaya, pectin, and tragacanth gums) and the like.

A glidant is a pharmaceutically acceptable excipient that eases powder flow of pharmaceutical mixtures. Suitable glidants are anhydrous colloidal silica (a.k.a. silicon dioxide), talc, magnesium carbonate, glyceryl behenate (including but not limited to Compritol® 888), hydrogenated vegetable oils (including but not limited to Sterotex®), waxes, boric acid, sodium benzoate, sodium acetate, sodium chloride, DL-leucine, sodium oleate and the like.

A lubricant is pharmaceutical excipient that prevents the ingredients from sticking to the tablet dies and punches. Suitable lubricants are sodium stearyl fumarate, stearic acid, magnesium stearate, calcium stearate, magnesium lauryl sulfate, talc, and the like.

A coating is defined here as a layer of a given thickness that surrounds the entire tablet surface.

A film coating is a thin layer (of about 0.02-0.5 mm) that surrounds a dosage form, here a tablet. The coating may perform different functions: aesthetic, ease the swallowing, modify the release of the drug (e.g. enteric coating, sustained release, etc). Suitable coatings include coatings based on hydroxypropylmethylcellulose (HPMC) or poly(vinyl alcohol) (PVA). Commercial examples are Opadry®, Opadry® 200, Opadry® amb II, Opadry® Fx™, Opadry® II, Opalux®.

Sugar coating involves the deposition from an aqueous solution of coatings based on sugars, typically sucrose.

A colorant is a product that provides colour to the pharmaceutical composition. Suitable colorants are: C.I. Pigment White 6, C.I. Natural Brown 10, C.I. Food Red 12, C.I. Food Red 17, C.I. Food Red 9, C.I. Food Red 3, C.I. Food Orange 8, C.I. Natural Red 4, C.I. Red 87, C.I. Food Red 14, C.I. Pigment Red 101 & 102, C.I. Food Red 7, C.I. Food Red 10, C.I. Food Orange 5, C.I. Food Orange 6, C.I. Natural Yellow 3, C.I. Food Yellow 13, C.I. Pigment Yellow 42 & 43, C.I. Food Yellow 13, C.I. Food Yellow 3, C.I. Food Yellow 4, C.I. Natural Green 3, C.I. Natural Green 3, C.I. Food Green 3, C.I. Food Green 4, C.I. Food Blue 2, C.I. Food Blue 1, C.I. Food Blue 5, C.I. Food Black 1, C.I. Pigment Black 11, C.I. Food Black 3 and the like.

A flavouring agent is a product that provides flavour to the pharmaceutical composition. Suitable flavouring agents are strawberry flavour, cherry flavour, banana flavour, mint flavour, orange, lemon, vanillin, peppermint, grape and the like.

A sweetener is an excipient that provides sweet taste to the pharmaceutical composition. Suitable sweeteners are preferably selected from the group consisting of sugars (such as sucrose, fructose, glucose and the like), artificial sweeteners (such as saccharin or its pharmaceutically acceptable salts (such as saccharin sodium), cyclamate or its pharmaceutically acceptable salts (such as cyclamate sodium), aspartame, acesulphame or its pharmaceutically acceptable salts (such as acesulphame potassium), sucralose, neohespiridin dihydrochalcone, naringin dihydrochalcone and the like), and mixtures thereof.

Further suitable excipients, definitions of the excipients described herein, examples and its role can be found on Handbook of Pharmaceutical Excipients, APhA Publications 6th edition 2009, edited by Raymond C. Rowe, Paul J. Sheskey and Marian E Quinn ISBN-978 0 85369 792 3. In this reference synonyms of the excipients cited here and further examples of specific types of excipients discussed here can be found.

A dry process for the manufacturing of a tablet is a process in which no solvent is used during the blending of the active ingredient and the excipients of the tablet core. Direct compression and dry compaction (including slugging and roller compaction) are dry processes used in the manufacture of tablets. If the tablet is further film coated, water or other solvents can be used in the film coating process.

Rivaroxaban has been described in at least three different polymorphs (crystalline modifications I, II and III) and some hydrates or solvates (see WO07039132 A1 for detailed description of rivaroxaban polymorphs and solvates). WO07039122 A2 states that crystalline modification form I is the thermodynamically stable form at room temperature with a DSC endotherm at 230° C. as prepared in example 1 of WO07039132 A1.

### DESCRIPTION OF THE FIGURES

FIG. 1 shows the dissolution profiles of examples E2, E3, E8, E14, E18, E21, E22 and E23 using the preferred dissolution method.

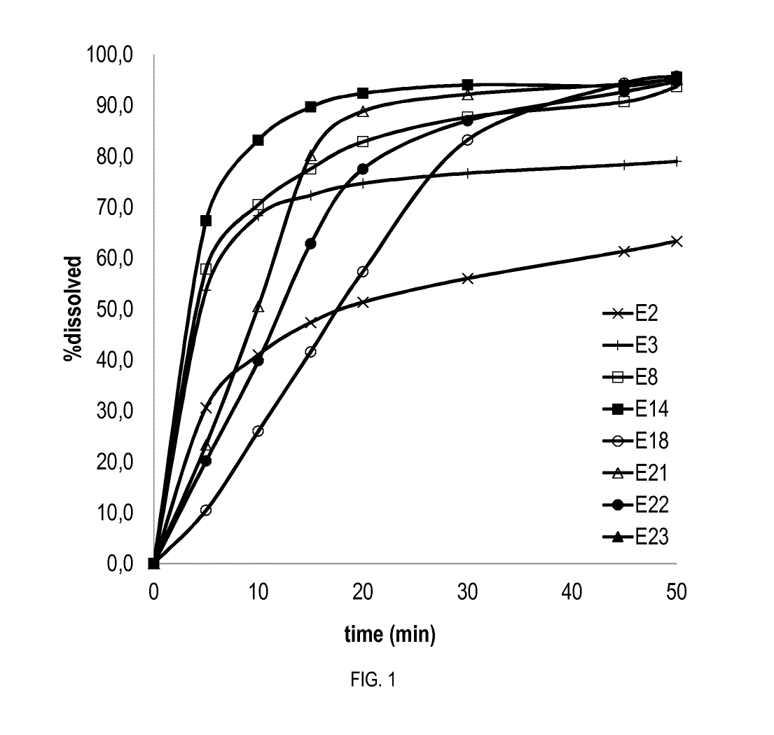

### DETAILED DESCRIPTION OF THE INVENTION

### Embodiment 1

An immediate release tablet comprising
rivaroxaban,
a non-ionic surfactant and
optionally one or more pharmaceutically acceptable excipients

obtainable by using a dry process.

### Embodiment 2

The tablet according to embodiment 1, wherein the non-ionic surfactant is selected from block copolymers of ethylene and propylene oxide, fatty acid esters, medium-chain triglycerides, cyclodextrins, polyethylene glycols, sorbitan alkyl esters, polyoxyethylene sorbitan fatty acid esters, polyoxylglycerides, polyoxyethylene fatty alcohol ethers, polyoxyethylene castor oil derivatives, polyoxyethylene alkyl esters, polyvinyl caprolactam-polyvinyl acetate-polyethylene glycol graft copolymer, polypropylene glycols esters or combinations thereof.

### Embodiment 3

The tablet according to embodiment 2, wherein the non-ionic surfactant is selected from block copolymers of ethylene and propylene oxide, polyvinyl caprolactam-polyvinyl acetate-polyethylene glycol graft copolymer or combinations thereof.

### Embodiment 4

The tablet according to embodiment 3, wherein the block copolymer of ethylene and propylene oxide is selected from poloxamer 124, poloxamer 188, poloxamer 237, poloxamer 331, poloxamer 338 and poloxamer 407 or combinations thereof.

### Embodiment 5

The tablet according to embodiment 4, wherein the block copolymer of ethylene and propylene oxide is poloxamer 407.

### Embodiment 6

The tablet according to embodiment 3, wherein the non-ionic surfactant is polyvinyl caprolactam-polyvinyl acetate-polyethylene glycol graft copolymer.

### Embodiment 7

The tablet according to embodiment 3, wherein the non-ionic surfactant is poloxamer 407.

### Embodiment 8

The tablet according to any of the previous embodiments, wherein the non-ionic surfactant is present in a 0.1 to 25% w/w with respect to the total tablet weight.

### Embodiment 9

The tablet according to embodiment 8, wherein the non-ionic surfactant is present in a 1-20% w/w with respect to the total tablet weight.

### Embodiment 10

The tablet according to embodiment 9, wherein the non-ionic surfactant is present in a 5 to 15% w/w with respect to the total tablet weight.

### Embodiment 11

The tablet according to any of the previous embodiments, wherein the non-ionic surfactant is solid.

### Embodiment 12

The tablet according to any of the previous embodiments, wherein the rivaroxaban used has a D90 value based on a volume distribution (Dv90) measured with laser light scattering lower than 25 μm.

### Embodiment 13

The tablet according to any of the previous embodiments, wherein the rivaroxaban used has a D90 value based on a volume distribution (Dv90) measured with laser light scattering lower than 20 μm.

### Embodiment 14

The tablet according to embodiments 12 or 13, wherein the rivaroxaban used has a D90 value based on a volume distribution (Dv90) measured with laser light scattering higher than 7 μm.

### Embodiment 15

The tablet according to any of the previous embodiments, wherein the rivaroxaban used is the thermodynamically stable crystalline at room temperature form with a DSC endotherm at 230° C.

### Embodiment 16

The tablet according to any of the previous embodiments, wherein rivaroxaban is the only drug present in the tablet.

### Embodiment 17

Tablets according to any of the previous embodiments wherein all the steps, except for the optional coating drying, are performed at room temperature.

### Embodiment 18

The tablet according embodiment 17, wherein all the steps, except for the optional coating drying, are performed at 15-30° C.

### Embodiment 19

The tablet according embodiment 18, wherein all the steps, except for the optional coating drying, are performed at 18-24° C.

### Embodiment 20

The tablet according to any of the previous embodiments, wherein the dry process is dry compaction, including roller compactation, slugging compactation, or direct compression.

### Embodiment 21

The tablet according to embodiment 20, wherein the dry process is dry compaction.

### Embodiment 22

The tablet according to embodiment 21, wherein the dry compaction is roller compaction.

### Embodiment 23

The tablet according to embodiment 21, wherein the dry compaction is slugging compaction.

### Embodiment 24

The tablet according to embodiment 20, wherein the dry process is direct compression.

### Embodiment 25

Tablets according to any of the previous embodiments wherein the particle size of rivaroxaban is not reduced during the process of manufacturing the tablets.

### Embodiment 26

Tablets according to any of the previous embodiments for the treatment or prevention of atherothrombotic events, prevention of venous thromboembolism, prevention of stroke and systemic embolism, treatment and prevention of deep vein thrombosis or treatment and prevention of pulmonary embolism.

### Embodiment 27

A process for the preparation of tablets according to embodiment 21, which comprises the following steps:

a) mixing rivaroxaban with at least the non-ionic surfactant,

b) compacting the mixture of step a),

c) sieving the compacted mixture of step b),

d) optionally adding the rest of the excipients and mixing, and

e) compressing the resulting mixture of steps c) or d) into a tablet,

wherein no liquid solvent is used in the process ending in step e).

### Embodiment 28

The process according to embodiment 27, wherein the compaction of step b) is a roller compaction

### Embodiment 29

The process according to embodiment 27, wherein the compaction of step b) is a slugging compaction.

### Embodiment 30

A process to prepare tablets according to embodiment 24, which comprises the following steps:

a) mixing rivaroxaban with at least the non-ionic surfactant,

b) optionally adding the rest of the excipients and mixing, and

c) compressing the resulting mixture into a tablet,

wherein no liquid solvent is used in the process ending in step c).

### Embodiment 31

The process to prepare tablets according to any of the embodiments 27 to 29, wherein steps a) to e) are performed at room temperature.

### Embodiment 32

the process to prepare tablets according to embodiment 30, wherein steps a) to c) are performed at room temperature.

### Embodiment 33

the process to prepare tablets according to any of the embodiments 31 or 32, wherein room temperature is between 15 and 30° C.

### Embodiment 34

the process to prepare tablets according to embodiment 33, wherein room temperature is between 18 and 24° C.

The following examples are illustrative and are not considered to limit the scope of the invention.

### EXAMPLES

Tablets are manufactured according to one of the following manufacturing examples:

CM: Fluid Bed Granulation (Comparative Example)

A granulation solution is prepared by dispersing the following components in purified water: rivaroxaban, Methocel® E5 premium LV and Kolliphor® SLS Fine. 72% of the total amount of Vivapur® 101, 62% of the total amount of Tablettose® 80 and all the amount of AC-DI-SOL® are introduced into the fluid bed granulator, they are mixed until an homogeneous mixture is obtained. Afterwards the mixture is granulated in the fluid bed granulator with the granulation solution prepared before according to the following conditions: inlet air temperature 65° C., outlet air temperature 28° C. and pressure of 2-3 bar. The resulting granules are dried in the same fluid bed dryer. Then the remaining amount of Vivapur® 101 and Tablettose® 80, and Ligamed® MF2V are added to the mixture of granules and mixed in a blender and finally compressed into tablets.

If desired, tablets can be film coated. In the quantitative composition it is stated which tablets have been film coated.

M1: Direct Compression

All the excipients, except for the Ligamed® MF2V and the Aerosil® 200, are blended with rivaroxaban. Afterwards Ligamed® MF2V and, if present, Aerosil® 200 are added to the mixture and further blended. Then the mixture is compressed into tablets.

If desired, tablets can be film coated. In the quantitative composition it is stated which tablets have been film coated.

M2: Dry Compaction (Slugging)

All the excipients, except for Ligamed® MF2V and Aerosil® 200, are blended with rivaroxaban. Afterwards Ligamed® MF2V and Aerosil® 200 are added to the mixture and further blended. Then the mixture is precompacted with a tablet press (slugging technique) and sieved through a mesh with sieve size of 1.0 mm. Afterwards the compacted blend is finally compressed into tablets.

If desired, tablets can be film coated. In the quantitative composition it is stated which tablets have been film coated.

M3: Dry Compaction (Roller Compaction)

All the excipients, except for Ligamed® MF2V, Aerosil® 200 and 15% of the content of Kollidon® CL are blended with rivaroxaban. Afterwards half of Ligamed® MF2V is added to the mixture and further blended. Then the mixture is compacted with a roller compactor and sieved through a mesh with sieve size of 1.0 mm. Then, the remaining Kollidon® CL and Aerosil® 200 are added and blended with the resulting mixture. Finally the remaining lubricant is added and the mixture is blended and finally compressed into tablets.

If desired, tablets can be film coated. In the quantitative composition it is stated which tablets have been film coated.

Rivaroxaban used in the examples was prepared according to WO0147919 A1 and optionally micronized, milled or sieved to the following particle size distribution with D90 values based on a volume distribution (Dᵥ90) measured by laser light scattering

R1 was micronized to a Dᵥ90 value of 14 μm.

R2 was micronized to a Dᵥ90 value of 15 μm.

R3 was micronized to a Dᵥ90 value of 19 μm.

R4 was milled to a Dᵥ90 value of 38 μm.

R5 was sieved to a Dᵥ90 value of 59 μm.

The following table includes the percentage in weight of rivaroxaban or excipients in relation to the total weight of the tablet core (% w/w or wt. %) of the compositions 01 to 020. The amount of film coat, when present, is not considered in weight of the tablet core and the amount of film coat is expressed as the percentage referring to the tablet core.

| Component | | Q1 | Q2 | Q3 | Q4 | Q5 | Q6 | Q7 |
|---|---|---|---|---|---|---|---|---|
| Rivaroxaban | | 23.53 | 17.72 | 11.76 | 11.76 | 11.76 | 11.76 | 11.76 |
| Soluplus ®* | | | | | 11.76 | 11.76 | 23.53 | 23.53 |
| Kolliphor ® P407 micro | Poloxamer 407 Micro | | | 23.53 | | | | |
| Vivapur ® 101 | Microcrystalline cellulose | | 43.36 | | | | | |
| | 101 | | | | | | | |
| Avicel ® PH102 | Microcrystalline cellulose | 41.18 | | 13.16 | 13.16 | 13.16 | 13.16 | 38.73 |
| | PH102 | | | | | | | |
| Tablettose ® 80 | Lactose monohydrate | 26.94 | 32.64 | 38.73 | 50.50 | 50.50 | 38.73 | 13.16 |
| AC-DI-SOL ® | Sodium Croscarmellose | 3.53 | 2.66 | | 5.12 | | 5.12 | 5.12 |
| Kollidon ® CL | Crospovidone | | | 5.12 | | 5.12 | | |
| Methocel ® E5 premium | Hypromellose 2910 E5 | 3.53 | 2.66 | 5.12 | 5.12 | 5.12 | 5.12 | 5.12 |
| LV | | | | | | | | |
| Kolliphor ® SLS fine | Sodium Laurylsulfate | 0.59 | 0.44 | 0.82 | 0.82 | 0.82 | 0.82 | 0.82 |
| Aerosil ® 200** | | | | 0.75 | 0.75 | 0.75 | 0.75 | 0.75 |
| Ligamed ® MF2V | Magnesium stearate | 0.71 | 0.53 | 1.00 | 1.00 | 1.00 | 1.00 | 1.00 |
| Total tablet | | 100.00 | 100.00 | 100.00 | 100.00 | 100.00 | 100.00 | 100.00 |
| Opadry II Pink | | | | | | | 3.00 | |
| 85F240050 | | | | | | | | |
| Opadry II Orange | | | | | | | | |
| 85F230047 | | | | | | | | |
| Opadry II Pink | | | | | | | | |
| 85F240118 | | | | | | | | |
| Total film coated | | | | | | | 103.00 | |
| Component | | Q8 | Q9 | Q10 | Q11 | Q12 | Q13 | Q14 |
| Rivaroxaban | | 11.76 | 11.76 | 11.76 | 11.76 | 11.76 | 23.53 | 11.76 |
| Soluplus ®* | | 23.53 | 23.53 | 47.06 | | | | |
| Kolliphor ® P407 micro | Poloxamer 407 Micro | | | | 1.18 | 1.18 | 5.88 | 11.76 |
| Vivapur ® 101 | Microcrystalline cellulose | | | | | | | |
| | 101 | | | | | | | |
| Avicel ® PH102 | Microcrystalline cellulose | 13.16 | 13.16 | 13.16 | 13.16 | 13.16 | 25.11 | 13.16 |
| | PH102 | | | | | | | |
| Tablettose ® 80 | Lactose monohydrate | 38.73 | 38.73 | 15.20 | 61.59 | 60.60 | 37.78 | 50.51 |
| AC-DI-SOL ® | Sodium Croscarmellose | 5.12 | 5.12 | 5.12 | | | | |
| Kollidon ® CL | Crospovidone | | | | 5.12 | 5.12 | 5.12 | 5.12 |
| Methocel ® E5 premium | Hypromellose 2910 E5 | 5.12 | 5.12 | 5.12 | 5.12 | 5.12 | | 5.12 |
| LV | | | | | | | | |
| Kolliphor ® SLS fine | Sodium Laurylsulfate | 0.82 | 0.82 | 0.82 | 0.82 | 0.82 | 0.82 | 0.82 |
| Aerosil ® 200** | | 0.75 | 0.75 | 0.75 | 0.75 | 0.75 | 0.75 | 0.75 |
| Ligamed ® MF2V | Magnesium stearate | 1.00 | 1.00 | 1.00 | 0.49 | 1.48 | 1.00 | 0.99 |
| Total tablet | | 100.00 | 100.00 | 100.00 | 100.00 | 100.00 | 100.00 | 100.00 |
| Opadry II Pink | | | | | | | | |
| 85F240050 | | | | | | | | |
| Opadry II Orange | | | | | | | | 3.00 |
| 85F230047 | | | | | | | | |
| Opadry II Pink | | | | | | | | |
| 85F240118 | | | | | | | | |
| Total film coated | | | | | | | | 103.00 |

| Component | | Q15 | Q16 | Q17 | Q18 | Q19 | Q20 |
|---|---|---|---|---|---|---|---|
| Rivaroxaban | | 11.76 | 11.76 | 11.76 | 11.76 | 11.76 | 11.76 |
| Soluplus ®* | | | | | | | |
| Kolliphor ® P407 micro | Poloxamer 407 Micro | 11.76 | 11.76 | 11.76 | 11.76 | 11.76 | 11.76 |
| Vivapur ® 101 | Microcrystalline cellulose 101 | | | | | | |
| Avicel ® PH102 | Microcrystalline cellulose PH102 | 13.16 | 13.16 | 13.16 | 13.16 | 13.16 | 13.16 |
| Tablettose ® 80 | Lactose monohydrate | 50.51 | 50.51 | 50.51 | 50.50 | 50.50 | 50.49 |
| AC-DI-SOL ® | Sodium Croscarmellose | | | | 5.12 | 5.12 | |
| Kollidon ® CL | Crospovidone | 5.12 | 5.12 | 5.12 | | | 5.12 |
| Methocel ® E5 premium LV | Hypromellose 2910 E5 | 5.12 | 5.11 | 5.12 | 5.12 | 5.12 | 5.12 |
| Kolliphor ® SLS fine | Sodium Laurylsulfate | 0.82 | 0.82 | 0.82 | 0.82 | 0.82 | 0.82 |
| Aerosil ® 200** | | 0.75 | 0.75 | 0.75 | 0.75 | 0.75 | 0.75 |
| Ligamed ® MF2V | Magnesium stearate | 0.99 | 0.99 | 0.99 | 1.00 | 1.00 | 1.00 |
| Total tablet | | 100.00 | 100.00 | 100.00 | 100.00 | 100.00 | 100.00 |
| Opadry II Pink 85F240050 | | 3.00 | | | | | |
| Opadry II Orange 85F230047 | | | | | | 3.00 | 3.00 |
| Opadry II Pink 85F240118 | | | 3.00 | | | | |
| Total film coated | | 103.00 | 103.00 | | | 103.00 | 103.00 |

*polyvinyl caprolactam-polyvinyl acetate-polyethylene glycol graft copolymer;
**Hydrophilic fumed silica with specific surface area of 200 m²/g.

20 mg rivaroxaban tablets were prepared with the following features:

| | Riva- | Manufacturing | Quantitative | Compression |
|---|---|---|---|---|
| Example | roxaban | method | composition | force (kN) |
| Comparative E1 | R3 | M1 | Q1 | 15 |
| Comparative E2 | R3 | M2 | Q1 | 15 |
| Comparative E3 | R3 | CM | Q2 | 15 |
| E4 | R2 | M1 | Q3 | 6 |
| E5 | R2 | M1 | Q4 | 13 |
| E6 | R2 | M2 | Q4 | 13 |
| E7 | R2 | M1 | Q5 | 10 |
| E8 | R2 | M1 | Q6 | 11 |
| E9 | R2 | M1 | Q7 | 7 |
| E10 | R2 | M2 | Q7 | 7 |
| E11 | R2 | M1 | Q8 | 10 |
| E12 | R2 | M2 | Q8 | 10 |
| E13 | R2 | M1 | Q10 | 15 |
| E14 | R1 | M3 | Q11 | 12 |
| E15 | R1 | M3 | Q12 | 11 |
| E16 | R3 | M2 | Q13 | 4 |
| E17 | R3 | M1 | Q13 | 4 |
| E18 | R1 | M3 | Q14 | 14 |
| E19 | R1 | M3 | Q15 | 13 |
| E20 | R1 | M3 | Q16 | 8 |
| E21 | R1 | M1 | Q17 | 10 |
| E22 | R4 | M1 | Q17 | 10 |
| E23 | R5 | M1 | Q17 | 10 |
| E24 | R2 | M1 | Q18 | 10 |
| E25 | R2 | M2 | Q18 | 10 |
| E26 | R2 | M1 | Q19 | 9 |
| E27 | R2 | M1 | Q20 | 6 |
| E28 | R2 | M2 | Q20 | 8 |
| E29 | R2 | M1 | Q9 | 4 |

Using the same rivaroxaban, methodology and quantitative composition, 2.5, 10 and 15 mg rivaroxaban tablets can be manufactured.

1. An immediate release tablet comprising
rivaroxaban,
a non-ionic surfactant, and
optionally one or more pharmaceutically acceptable excipients
wherein the immediate release tablet is obtained using a dry process.

2. The tablet according to claim 1, wherein the non-ionic surfactant is selected from block copolymers of ethylene and propylene oxide, fatty acid esters, medium-chain triglycerides, cyclodextrins, polyethylene glycols, sorbitan alkyl esters, polyoxyethylene sorbitan fatty acid esters, polyoxylglycerides, polyoxyethylene fatty alcohol ethers, polyoxyethylene castor oil derivatives, polyoxyethylene alkyl esters, polyvinyl caprolactam-polyvinyl acetate-polyethylene glycol graft copolymer, polypropylene glycols esters or combinations thereof.

3. The tablet according to claim 2, wherein the non-ionic surfactant is selected from block copolymers of ethylene and propylene oxide, polyvinyl caprolactam-polyvinyl acetate-polyethylene glycol graft copolymer or combinations thereof.

4. The tablet according to claim 3, wherein the block copolymer of ethylene and propylene oxide is poloxamer 407.

5. The tablet according to claim 3, wherein the non-ionic surfactant is polyvinyl caprolactam-polyvinyl acetate-polyethylene glycol graft copolymer.

6. The tablet according to claim 3, wherein the non-ionic surfactant is poloxamer 407.

7. The tablet according to claim 1, wherein the non-ionic surfactant is present in a 0.1 to 25% w/w with respect to the total tablet weight.

8. The tablet according to claim 7, wherein the non-ionic surfactant is present in a 5 to 15% w/w with respect to the total tablet weight.

9. The tablet according to claim 1, wherein the rivaroxaban used has a D90 value based on a volume distribution (Dᵥ90) measured with laser light scattering lower than 25 μm.

10. The tablet according to claim 9, wherein the rivaroxaban used has a D90 value based on a volume distribution (Dᵥ90) measured with laser light scattering higher than 7 μm.

11. The tablet according to claim 1, wherein the dry process is dry compaction.

12. The tablet according to claim 1, wherein the dry process is direct compression.

13. Tablets according to claim 1 for the treatment or prevention of atherothrombotic events, prevention of venous thromboembolism, prevention of stroke and systemic embolism, treatment and prevention of deep vein thrombosis or treatment and prevention of pulmonary embolism.

14. A process for the preparation of tablets according to claim 11, which comprises the following steps:
a) mixing rivaroxaban with at least the non-ionic surfactant,
b) compacting the mixture of step a),
c) sieving the compacted mixture of step b),
d) optionally adding the rest of the excipients and mixing, and
e) compressing the resulting mixture of steps c) or d) into a tablet,
wherein no liquid solvent is used in the process ending in step e).

15. A process to prepare tablets according to claim 12, which comprises the following steps:
a) mixing rivaroxaban with at least the non-ionic surfactant,
b) optionally adding the rest of the excipients and mixing, and
c) compressing the resulting mixture into a tablet,
wherein no liquid solvent is used in the process ending in step c).

16. The tablet according to claim 2, wherein the non-ionic surfactant is present in a 0.1 to 25% w/w with respect to the total tablet weight.

17. The tablet according to claim 3, wherein the non-ionic surfactant is present in a 0.1 to 25% w/w with respect to the total tablet weight.

18. The tablet according to claim 4, wherein the non-ionic surfactant is present in a 0.1 to 25% w/w with respect to the total tablet weight.

19. The tablet according to claim 5, wherein the non-ionic surfactant is present in a 0.1 to 25% w/w with respect to the total tablet weight.

20. The tablet according to claim 6, wherein the non-ionic surfactant is present in a 0.1 to 25% w/w with respect to the total tablet weight.
